(19) United States
(12) Patent Application Publication
(10) Pub. No.: US 2010/0059055 A1
(43) Pub. Date: Mar. 11, 2010

(54) Gas delivery system including a flow generator having an isolated blower assembly for noise reduction

(76) Inventors: Timothy A. Brungart, State College, PA (US); Eric C. Myer, Spring Mills, PA (US); Adam N. Baxter, Pittsburgh, PA (US); Richard Alfieri, Delmont, PA (US); Chris Gorman, Pittsburgh, PA (US)

Correspondence Address: BUCHANAN INGERSOLL & ROONEY PC, P.O. BOX 1404, ALEXANDRIA, VA, 22313-1404, US

(21) Appl. No.: 12/231,820

(22) Filed: Sep. 5, 2008

(51) Int. Cl. A61M 16/10

(52) U.S. Cl. 128/204.18

(57) ABSTRACT

A gas delivery system that includes a housing and a flow generator that includes a blower assembly and an isolation assembly for coupling the flow generator to the housing. The isolation assembly includes at least one elastomeric isolation member having first and second portions, and the blower assembly is coupled to the isolation assembly through the at least one elastomeric isolation member. The first portion is able to shear in a first direction and the second portion is able to shear in a second direction generally perpendicular to the first direction to permit the blower assembly to move relative to the housing in three dimensions. Also, a gas delivery system that includes a housing and a flow generator provided within the housing, wherein the gas delivery system generates no more than about 30 or 35 dB(A) of noise regardless of the physical orientation of the housing.

### BACKGROUND OF THE INVENTION

1. Field of the Invention

The present invention relates to gas delivery systems, and, in particular, to a gas delivery system having a flow generator that includes an isolation assembly for isolating the flow generator from an enclosure of the gas delivery system in order to reduce vibration and noise.

2. Description of the Related Art

Medical devices that provide a flow of gas to an airway of a patient are used in a variety of situations. For example, ventilators replace or augment a patient's own breathing, pressure support devices deliver pressurized gas to treat breathing disorders, such as obstructive sleep apnea (OSA), and anesthesia machines deliver an anesthesia gas to the patient. For purposes of the present invention, any such device that delivers a flow of gas to the airway of the patient, invasively or non-invasively, is referred to herein as a gas delivery system.

These devices include a flow generator for generating the flow of gas that is delivered to the patient. A typical flow generator may include a brushless electric motor driving an impeller, which is often referred to in combination as a blower or blower assembly.

During operation, vibrations that are caused by the blower assembly may cause noise to be generated by the gas delivery system in which the flow generator is mounted. Additionally, the air drawn into the gas delivery system to an inlet associated with the flow generator may also cause operating noise. Treatment provided by gas delivery systems are often delivered to the patient while the patient, and any bed partners, are sleeping (or attempting to sleep). Consequently, minimizing sound emission from a gas delivery system is of significant concern. Any noise can serve to disrupt the patient's sleep, or the sleep of others, and should be minimized.

Conventional attempts to minimize the operating noise caused by the flow generator within a gas delivery system have proved ineffective, inefficient, and/or expensive. For example, some existing gas delivery systems have utilized sound insulating materials, such as foam, in the housing construction. Insulation materials such as those used in the prior art are able to reduce noise. However, the use of such insulation materials becomes difficult with smaller product profiles. In other words, as gas delivery systems are being made smaller, the thicknesses of the insulation materials is decreased and therefore the effectiveness is reduced. Therefore, a need exists for a mounting assembly for mounting a flow generator within a gas delivery system that effectively and efficiently reduces operating vibration and noise caused by the flow generator.

### SUMMARY OF THE INVENTION

Accordingly, it is an object of the present invention to provide a gas delivery system that overcomes the shortcomings of conventional gas delivery systems. This object is achieved according to one embodiment of the present invention by providing, in one embodiment, a gas delivery system that includes a housing and a flow generator. The flow generator includes a blower assembly and an isolation assembly for coupling the flow generator to the housing. The isolation assembly includes at least one elastomeric isolation member, and the blower assembly is coupled to the isolation assembly through the at least one elastomeric isolation member. The at least one elastomeric isolation member has a first portion and a second portion. The first portion is structured to be able to shear in a first direction and the second portion is structured to be able to shear in a second direction generally perpendicular to the first direction to permit the blower assembly to move relative to the housing in three dimensions. When the first portion shears in the first direction, the second portion compresses in the first direction, and when the second portion shears in the second direction, the first portion compresses in the second direction. In addition, the first portion may also be structured to be able to shear about an axis of rotation of a motor of the blower assembly, and the second portion may also be structured to be able to shear about an axis substantially perpendicular to the motor's axis of rotation.

In one particular embodiment, the at least one elastomeric isolation member includes a first elastomeric isolation member structured to be able to shear in the first direction and a second elastomeric isolation member separate from the first elastomeric isolation member and structured to be able to shear in the second direction. The first elastomeric isolation member and the second elastomeric isolation member may each have a generally annular shape. Alternatively, the at least one elastomeric isolation member may be a single member having the portions which shear as described.

The blower assembly may include a motor operatively coupled to an impeller. Further, the blower assembly may include a blower housing, wherein the impeller is provided within blower housing, wherein a surface (e.g., top surface) of the impeller has a first shape, and wherein a portion (e.g., an upper housing portion) of the blower housing has a second shape that substantially matches the first shape. In one particular embodiment, the impeller and motor components that rotate about the motor's axis of rotation have a mass moment of inertia between about 0.9 lb-in² and about 1.3 lb-in². In another particular embodiment, the impeller and rotating motor components have a mass moment of inertia that is less than or equal to about 1.3 lb-in². In still another particular embodiment, the impeller has a radius of between about 19 mm and about 37 mm.

Furthermore, the flow generator may include an elastomeric bellows member for coupling the air inlet of the blower housing to the air inlet of the gas delivery system. The flow generator may also include an elastomeric tube for coupling the flow outlet of the blower housing to a patient gas delivery circuit of the gas delivery system.

Another embodiment provides a gas delivery system that includes a housing and a flow generator provided within the housing, wherein the gas delivery system generates no more than about 35 dB(A) of noise in the case of, for example, a portable ventilator, and no more than about 30 dB(A) of noise in the case of, for example, a CPAP machine regardless of the physical orientation of the housing.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIG. 1 is an isometric view of a flow generator according to one embodiment of the present invention;

FIG. 2 is a cross-sectional view of the flow generator shown in FIG. 1;

FIG. 3 is an exploded view of the flow generator shown in FIG. 1;

FIG. 4 is an isometric view of the isolation assembly used to support the flow generator shown in FIG. 1;

FIG. 5 is an isometric view of an isolation housing forming a part of the isolation assembly shown in FIG. 4;

FIG. 6 is an isometric view of a blower mounting component forming a part of the isolation assembly shown in FIG. 4;

FIG. 7 is an isometric view of a first isolator attachment part forming a part of the isolation assembly shown in FIG. 4;

FIG. 8 is an isometric view showing a partially assembled isolation assembly shown in FIG. 4;

FIG. 9 is an isometric view of a second isolator attachment part forming a part of the isolation assembly shown in FIG. 4;

FIG. 10 is a isometric view of an elastomeric tube assembly forming a part of the flow generator shown in FIG. 1;

FIG. 11 is a isometric view of an elastomeric bellows member forming a part of the flow generator shown in FIG. 1; and

FIG. 12 is an isometric view of an exemplary ventilator in which the flow generator shown in FIG. 1 may be used.

### DESCRIPTION OF THE EXEMPLARY EMBODIMENTS

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

As employed herein, the statement that two or more parts or components are “coupled” together shall mean that the parts are joined or operate together either directly or through one or more intermediate parts or components.

As employed herein, the term “number” shall mean one or an integer greater than one (i.e., a plurality).

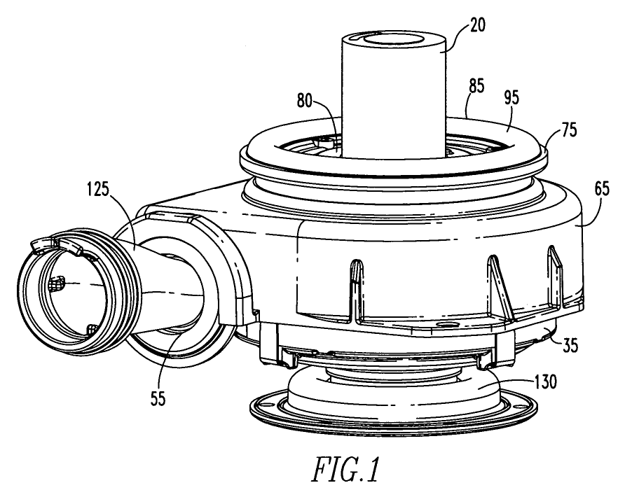

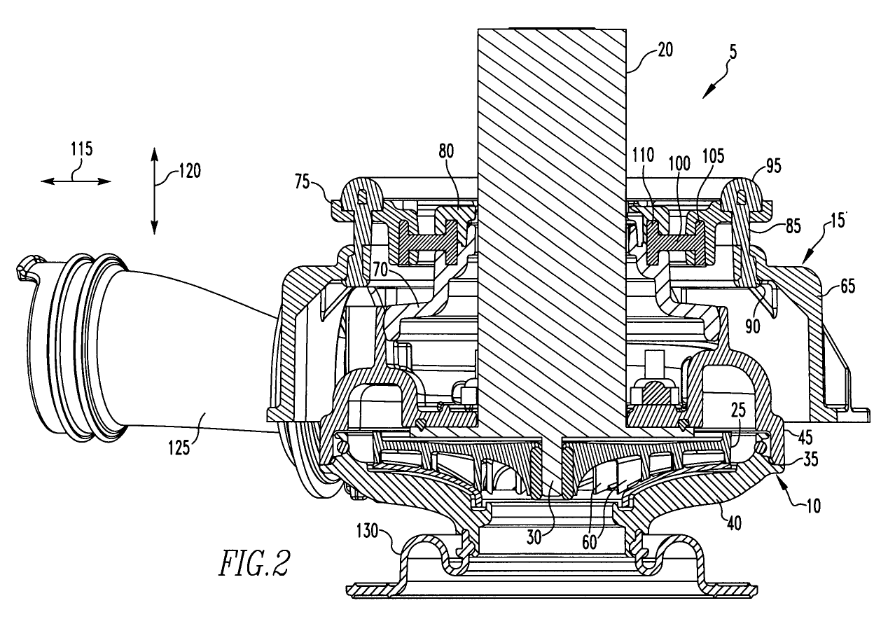

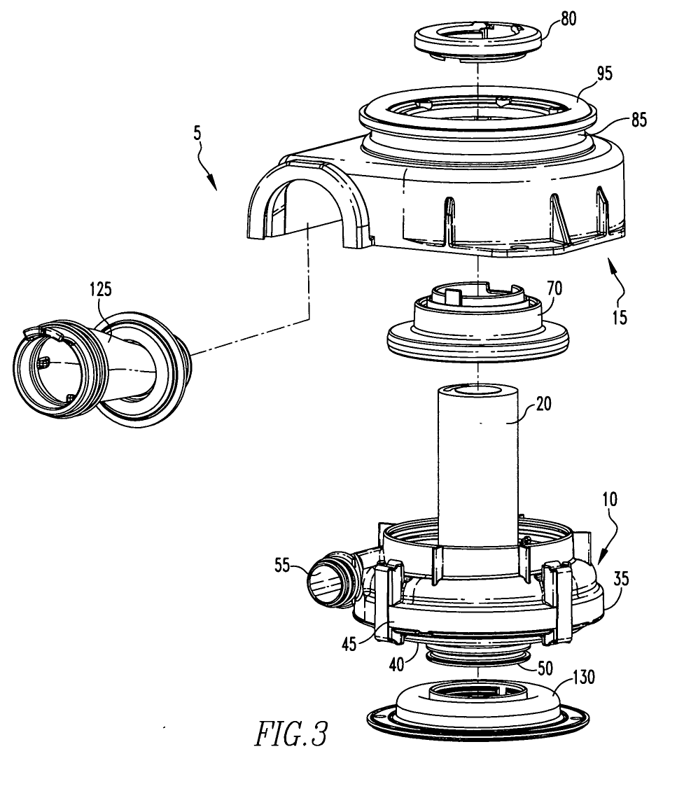

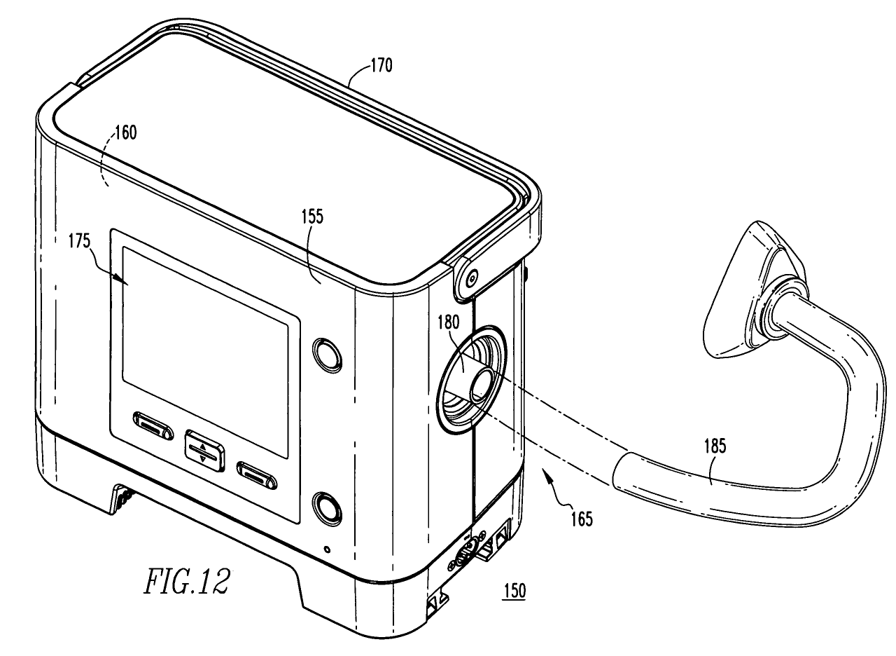

FIG. 1 is an isometric view of a flow generator 5 according to one embodiment of the present invention. FIG. 2 is a cross-sectional view of the flow generator 5 shown in FIG. 1, and FIG. 3 is an exploded view of the flow generator 5 shown in FIG. 1. Flow generator 5 is structured for use in a gas delivery system in order to generate a flow of gas for delivery to the airway of a patient. For example, and without limitation, flow generator 5 may be used in a medical ventilator 150 as shown in FIG. 12. Medical ventilator 150 includes a housing 155 having an interior 160, and an exterior 165. The exemplary ventilator 150 is designed to be portable and, therefore, includes a handle 170, which is pivotably coupled to the top of the housing 155 and shown in a “folded down” or retracted position in FIG. 12, in order to facilitate carrying or moving of ventilator 150.

In the example of FIG. 12, ventilator 150 includes a user interface 175, which is disposed on the exterior surface at the front of ventilator housing 155. Finally, housing 155 of ventilator 150 includes an inlet port (not shown) for supplying air or another gas to flow generator 5, as described elsewhere herein, and an outlet port 180 structured to be coupled to a patient gas delivery circuit 185 for delivering the flow of gas generated by the flow generator to the airway of a patient.

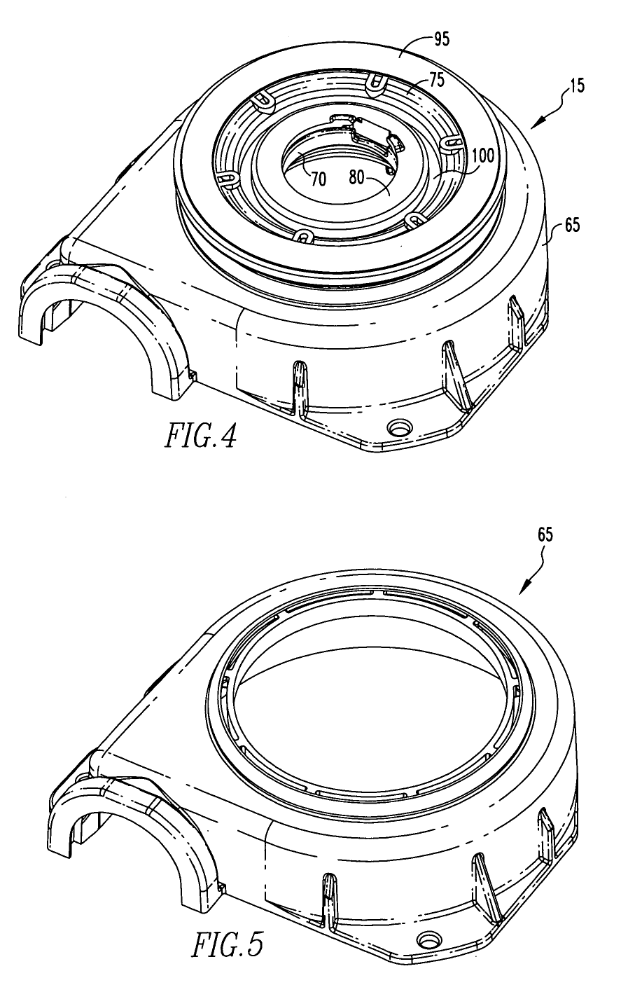

Referring again to FIGS. 1, 2 and 3, flow generator 5 includes a blower assembly 10 coupled to an isolation assembly 15 (shown in isometric view in FIG. 4). Isolation assembly 15 is used to mount flow generator 5 within a housing or enclosure of a gas delivery system, such as, without limitation, a ventilator, a pressure support device or an anesthesia machine (e.g., housing 155 of ventilator 150 shown in FIG. 12). In addition, as described in greater detail herein, isolation assembly 15 reduces vibration of the gas delivery system and associated acoustic noise caused by the vibration of blower assembly 10.

As is common in the art, the housings and mounting elements provided therein (such as bedplates) of gas delivery systems are typically made of rigid materials, such as rigid injection molded polymers. Such rigidity is necessary to provide the structural support and structural integrity for the gas delivery system and the components thereof. However, a rigid structure easily transmits vibrations therethrough, which may result in significant noise. Isolation assembly 15 isolates blower assembly 10 from the housing of the gas delivery system in which flow generator 5 is mounted in order to minimize the vibration that is transmitted thereto and thereby minimize the associated acoustic noise. Isolation assembly 15 (and therefore flow generator 5) may be directly coupled to the housing, or indirectly coupled to the housing through, for example, a rigid bedplate provided within the housing and/or one or more other intermediate mounting elements provided within the housing.

As most readily seen in FIG. 2, blower assembly 10 includes a motor 20 (e.g., a brushless electric motor) which is operatively coupled to an impeller 25 (or multiple impellers) through a shaft 30. Impeller 25 and shaft 30 are enclosed within a blower housing 35; including an upper housing portion 40 and a lower housing portion 45. Blower housing 35 defines an air inlet 50 and a flow outlet 55 (FIG. 3). Impeller 25 includes a plurality of blades 60 such that when the impeller 25 is rotatably driven by motor 20, blades 60 force air contained within blower housing 35 to exit the blower housing through flow outlet 55. As the air in blower housing 35 is forced out of flow outlet 55, air is drawn into blower housing 35 through air inlet 50.

In the illustrated exemplary embodiment, impeller 25 has a radius of between about 19 mm and about 37 mm, and in a further embodiment, about 26.5 mm. Also, in an exemplary embodiment, impeller 25 and rotating components of motor 20 have a mass moment of inertia of between about 0.9 lb-in² and 1.3 lb-in², and most particularly, no more than about 1.1 lb-in². Moreover, as seen in FIG. 2, the shape of the top surface of impeller 25 according to an exemplary embodiment substantially matches the shape of lower housing portion 45, which shape is preferably a curved shape, as seen in FIG. 2.

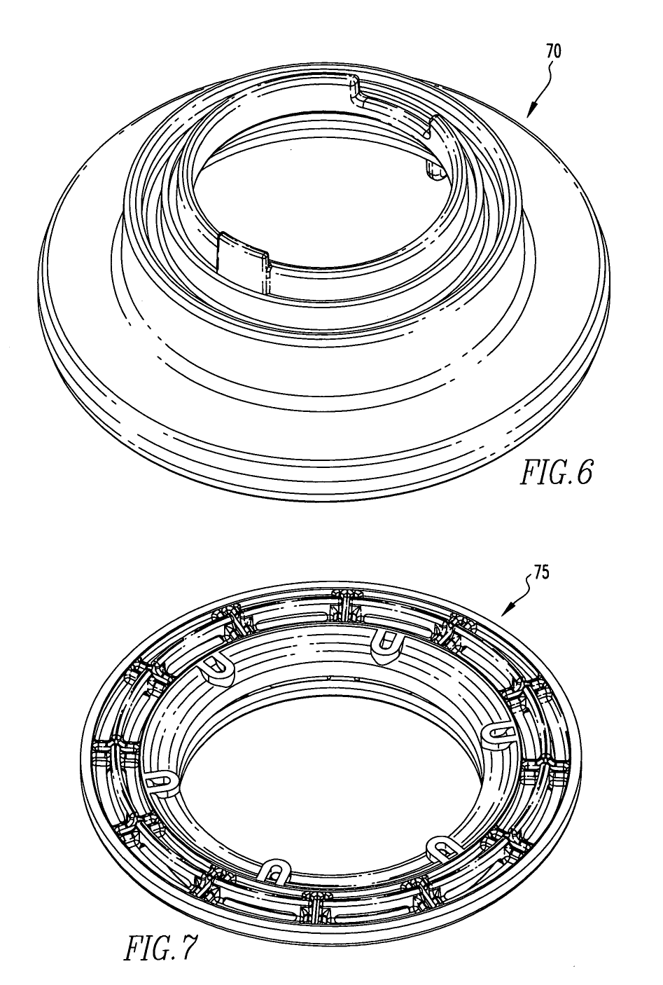

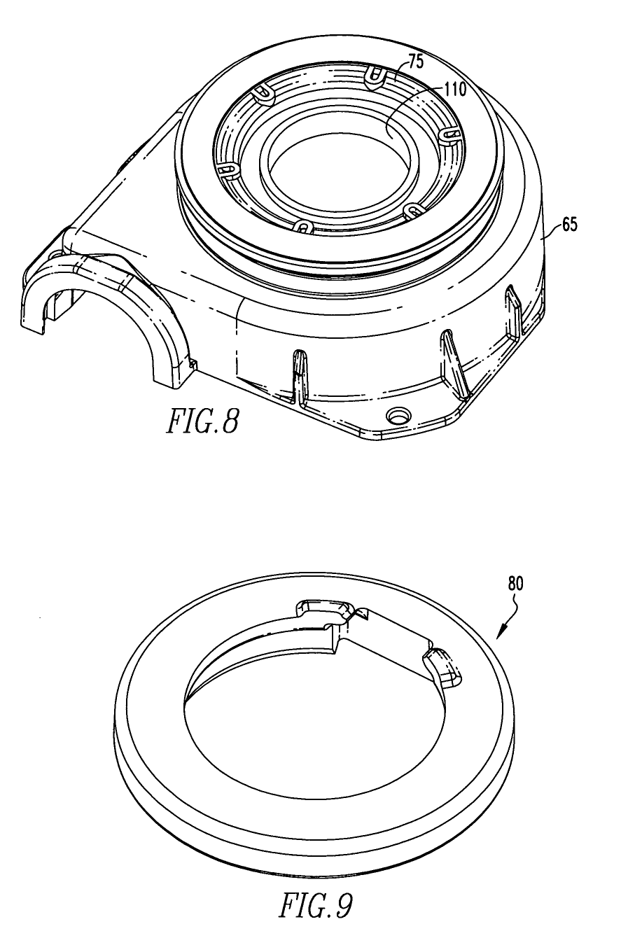

Isolation assembly 15, which is shown in cross section in FIG. 2 as part of the flow generator 5 and alone in isometric view in FIG. 4, includes an isolation housing 65 (FIG. 5) that, in an exemplary embodiment, is made of a rigid material, such as an injection molded polymer. Isolation housing 65 is used to mount flow generator 5 to the housing of the gas delivery system in which flow generator 5 is provided. Isolation assembly 15 further includes a blower mounting component 70 (FIG. 6), a first isolator attachment part 75 (FIG. 7 and FIG. 8, shown mounted with the isolation housing 65) and a second isolator attachment part 80 (FIG. 9). Blower mounting component 70, first isolator attachment part 75, and second isolator attachment part 80 are each preferably made of a rigid material, such as an injection molded polymer.

Isolation assembly 15 also further includes a first generally annular elastomeric isolation member 85 having a first end 90 and a second end 95, and a second generally annular elastomeric isolation member 100 having a first end 105 and a second end 110. First and second generally annular elastomeric isolation members 85 and 100 are each made of a flexible material, such as, without limitation, a rubber material or a rubber-like polymer material. We have found that 20 durometer silicon rubber works well. We prefer that both isolation members be made of the same material in applications such as the one shown in the drawings. However, the first isolation member 85 could be made of a different elastomeric material than the second isolation member 100. The function and importance of the first and second generally annular elastomeric isolation members 85 and 100 is described in detail below. Although generally annular elastomeric isolation members 85 and 100 are employed in the embodiment shown in FIGS. 1-3, it should be understood that this is meant to be exemplary only and should not be considered limiting, as other shapes may also be employed within the scope of the present invention.

As shown in FIG. 2, blower mounting component 70 is structured to connect blower assembly 10 to isolation assembly 15 by mating with and attaching to (via a friction, a snap fit or some other suitable attaching method) lower housing portion 45 of blower housing 35. In addition, when isolation assembly 15 is assembled, first isolation member 85 is positioned between isolation housing 65 and first isolator attachment part 75.

Specifically, first end 90 of first isolation member 85 is received within a groove provided in isolation housing 65, and second end 95 of the first isolation member is received through and held by the outer perimeter of first isolator attachment part 75. In addition, second isolation member 100 is positioned between first isolator attachment part 75, blower mounting component 70, and second isolator attachment part 80. Specifically, first end 105 of second isolation member 100 is received within and held by a groove provided in the inner perimeter of first isolator attachment part 75, and second end 110 of second isolation member 100 is received within and held by a grooves provided in both blower mounting component 70 and second isolator attachment part 80 after it has been fastened over motor 20 (the motor is inserted through the hole in the center of second isolator attachment part 80 and blower mounting component 70).

First and second generally annular elastomeric isolation members 85 and 100 isolate blower assembly 10 from the housing of the gas delivery system in which flow generator 5 is mounted by allowing blower assembly 10 to move relative to isolator housing 65 in three dimensions. In particular, first isolation member 85 is able to shear in a direction that is substantially perpendicular to the axis of rotation of motor 20 as shown by arrows 115 in FIG. 2 and about the axis of rotation of motor such that first end 90 and second end 95 thereof are able to move relative to one another in parallel planes. The second isolation member 100 is able to shear in the direction that is substantially parallel to the axis of rotation of motor 20 as shown by arrows 120 in FIG. 2, and about an axis that is perpendicular to the axis of rotation of the motor such that first end 105 and second end 110 thereof are able to move relative to one another in parallel planes. In other words, first isolation member 85 and second isolation member 100 work in different planes. Preferably, the directions of the shearing that is permitted are generally perpendicular to one another.

As a result of the structure described above, isolation assembly 15 allows for three dimensions of movement of blower assembly 10 in order to reduce the transmission of vibrations from the blower assembly to the housing of the gas delivery system in which it is provided and thereby reduce noise. Isolation, which provides for three dimensions movement as described herein, is significant and advantageous because it allows noise to be reduced regardless of the directional orientation of the gas delivery system in which flow generator 5 is mounted. The present invention allows noise generation to be kept below some threshold level regardless of whether the gas delivery system is oriented upside down, right side up, or on its side. In a portable ventilator (e.g., ventilator 150 shown in FIG. 12) the noise generated by a gas delivery system can be kept below 35 dB(A) (measured at 1 meter). The present invention can maintain noise generated by the gas delivery system in a CPAP machine at not more than 30 dB(A).

While first and second isolation members 85, 100 are shown in the exemplary embodiment, it should be understood that this is not meant to be limiting. For example, a single elastomeric isolation member may be provided as part of isolation assembly 15 that includes separate portions that are able to shear in the directions described above in order to provide the three dimensions of movement as described herein.

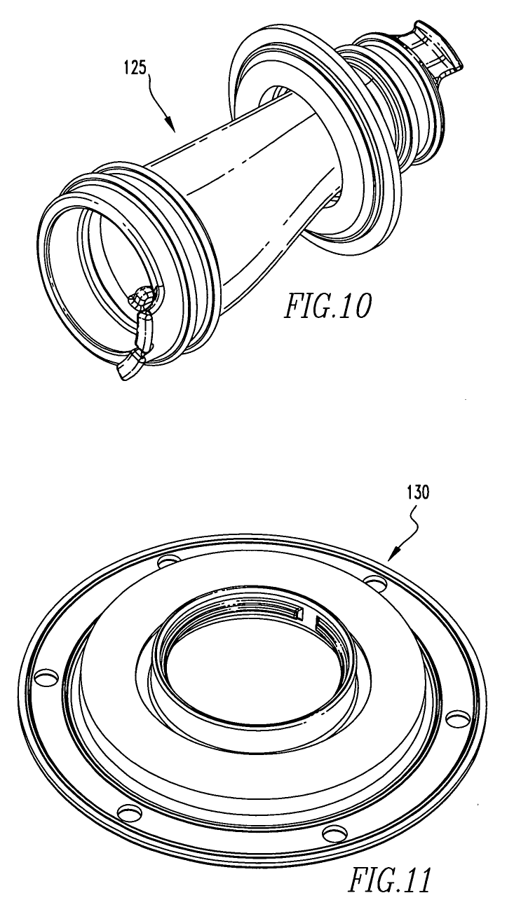

According to a further aspect of the exemplary embodiment shown in FIGS. 1-9, an elastomeric tube 125 (FIG. 10) is operatively coupled to flow outlet 55 in order to connect the flow outlet to a patient gas delivery circuit (e.g., patient gas delivery circuit 185 through outlet port 180 of ventilator 150 shown in FIG. 12) so that the flow of gas generated by flow generator 5 can be delivered to the patient. Because tube 125 is made of an elastomeric material, it further helps to isolate blower assembly 10 from the housing (e.g., housing 155) of the gas delivery system in which the flow generator is mounted and therefore further reduces noise.

According to still a further aspect of the exemplary embodiment shown in FIGS. 1-9, first end of an elastomeric bellows member 130 (FIG. 11) is operatively coupled to air inlet 50. The second end of bellows member 130 is, in turn, operatively coupled to the air inlet forming a part of the housing of the gas delivery system in which flow generator 5 is mounted (e.g., housing 155 of ventilator 150 shown in FIG. 12) so that a source of gas can be provided to air inlet 50 through the bellows member. Because bellows member 130 is made of an elastomeric material, it is able to deform in the direction of arrows 115 and 126 shown in FIG. 2 to further isolate blower assembly 10 from the housing (e.g., housing 155) of the gas delivery system in which flow generator 5 is mounted and therefore further reduce noise.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A gas delivery system, comprising:
a housing; and
a flow generator comprising a blower assembly and an isolation assembly adapted to couple the flow generator to the housing, wherein the isolation assembly includes at least one elastomeric isolation member, wherein the blower assembly is coupled to the isolation assembly through the at least one elastomeric isolation member, and wherein the at least one elastomeric isolation member has a first portion and a second portion, the first portion being structured to be able to shear in a first direction and the second portion being structured to be able to shear in a second direction substantially perpendicular to the first direction to permit the blower assembly to move relative to the housing in three dimensions.

2. The gas delivery system according to claim 1, wherein when the first portion shears in the first direction, the second portion compresses in the first direction, and wherein when the second portion shears in the second direction, the first portion compresses in the second direction.

3. The gas delivery system according to claim 1, wherein the at least one elastomeric isolation member includes a first elastomeric isolation member structured to be able to shear in the first direction and a second elastomeric isolation member separate from the first elastomeric isolation member and structured to be able to shear in the second direction, the first elastomeric isolation member being the first portion and the second elastomeric isolation member being the second portion.

4. The gas delivery system according to claim 3, wherein the first elastomeric isolation member and the second elastomeric isolation member each have a generally annular shape.

5. The gas delivery system according to claim 1, wherein the blower assembly includes a motor operatively coupled to at least one impeller, the motor having an axis of rotation, the first direction being substantially perpendicular to the axis of rotation.

6. The gas delivery system according to claim 5, wherein the first portion is also structured to be able to shear about the axis of rotation, and wherein the second portion is also structured to be able to shear about an axis substantially perpendicular to the axis of rotation.

7. The gas delivery system according to claim 5, wherein the blower assembly includes a blower housing, wherein the at least one impeller is provided within the blower housing, wherein a surface of the at least one impeller has a first shape, and wherein a portion of the blower housing has a second shape that substantially matches the first shape.

8. The gas delivery system according to claim 7, wherein the surface of the at least one impeller is a top surface of the at least one impeller, wherein the blower housing comprises an upper housing portion coupled to a lower housing portion, and wherein the portion of the blower housing is the upper housing portion.

9. The gas delivery system according to claim 5, wherein the motor includes rotating components, wherein the at least one impeller and the rotating components have a mass moment of inertia between about 0.9 lb-in² and about 1.3 lb-in².

10. The gas delivery system according to claim 5, wherein the motor includes rotating components, wherein the at least one impeller and the rotating components have a mass moment of inertia that is less than or equal to about 1.3 lb-in².

11. The gas delivery system according to claim 5, wherein the at least one impeller has a radius of between about 19 mm and about 37 mm.

12. The gas delivery system according to claim 1, wherein the blower assembly includes a blower housing having an air inlet and a flow outlet, wherein the gas delivery system includes an air inlet, and wherein the flow generator includes an elastomeric bellows member for coupling the air inlet of the blower housing to the air inlet of the gas delivery system.

13. The gas delivery system according to claim 1, wherein the blower assembly includes a blower housing having an air inlet and a flow outlet, and wherein the flow generator includes an elastomeric tube for coupling the flow outlet of the blower housing to a patient gas delivery circuit of the gas delivery system.

14. The gas delivery system according to claim 1, wherein the isolation assembly includes an isolation housing attached to the housing of the gas delivery system, and wherein the blower assembly is coupled to the isolation housing through the at least one elastomeric isolation member.

15. A gas delivery system, comprising:
a housing; and
a flow generator comprising a blower assembly and an isolation assembly for coupling the flow generator to the housing, wherein the isolation assembly includes means for coupling the blower assembly to the isolation assembly in a manner that permits the blower assembly to move relative to the housing in three dimensions.

16. The gas delivery system according to claim 15, wherein the blower assembly includes a motor operatively coupled to at least one impeller.

17. The gas delivery system according to claim 16, wherein the blower assembly includes a blower housing, wherein the at least one impeller is provided within the blower housing, wherein a surface of the at least one impeller has a first shape, and wherein a portion of the blower housing has a second shape that substantially matches the first shape.

18. The gas delivery system according to claim 17, wherein the surface of the impeller is a top surface of the impeller, wherein the blower housing comprises an upper housing portion coupled to a lower housing portion, and wherein the portion of the blower housing is the upper housing portion.

19. The gas delivery system according to claim 16, wherein the motor includes rotating components, wherein the at least one impeller and the rotating components have a mass moment of inertia between about 0.9 lb-in² and about 1.3 lb-in².

20. The gas delivery system according to claim 16, wherein the motor includes rotating components, wherein the at least one impeller and the rotating components have a mass moment of inertia that is less than or equal to about 1.3 lb-in².

21. The gas delivery system according to claim 16, wherein the at least one impeller has a radius of between about 19 mm and about 37 mm.

22. The gas delivery system according to claim 15, wherein the blower assembly includes a blower housing having an air inlet and a flow outlet, wherein the gas delivery system includes an air inlet, and wherein the flow generator includes means for flexibly coupling the air inlet of the blower housing to the air inlet of the gas delivery system.

23. The gas delivery system according to claim 15, wherein the blower assembly includes a blower housing having an air inlet and a flow outlet, and wherein the flow generator includes means for flexibly coupling the flow outlet of the blower housing to a patient gas delivery circuit of the gas delivery system.

24. The gas delivery system according to claim 15, wherein the isolation assembly includes an isolation housing attached to the housing of the gas delivery system, and wherein the means for coupling the blower assembly to the isolation assembly comprises means for coupling the blower assembly to the isolation housing in a manner that permits the blower assembly to move relative to the isolation housing in three dimensions.

25. A gas delivery system, comprising:
a housing; and
a flow generator provided within the housing, the gas delivery system generating no more than about 35 dB(A) of noise regardless of the physical orientation of the housing.

26. The gas delivery system according to claim 25, the gas delivery system generating no more than about 30 dB(A) of noise regardless of the physical orientation of the housing.
